(19) United States
(12) Patent Application Publication
(10) Pub. No.: US 2015/0258288 A1
(43) Pub. Date: Sep. 17, 2015

(54) Breathable Fluid Delivery System Including Exchangeable Fluid Permeable Cartridge

(71) Applicant: VOODOO SCIENCE LLC, Los Angeles, CA (US)

(72) Inventor: Nicholas Grayson Sullivan, La Canada Flintridge, CA (US)

(73) Assignee: Voodoo Science LLC, Los Angeles, CA (US)

(21) Appl. No.: 14/203,560

(22) Filed: Mar. 11, 2014

(51) Int. Cl. A61M 15/00; A24F 47/00

(52) U.S. Cl. CPC A61M 15/0086; A61M 15/002; A24F 47/008

(57) ABSTRACT

The present invention provides improved apparatus, systems, and methods of delivering a breathable fluid stream carrying an atomized or vaporized functional liquid. In particular, a fluid-permeable exchangeable cartridge is provided for containing the functional liquid to be introduced into a fluid stream comprising a breathable base fluid flowing through the cartridge. Preferably, the breathable base fluid includes a solvation enhancer such as atomized or vaporized propylene glycol or glycerin. In another aspect, a throttling valve system is provided for adjusting the ratio of flow from a breathable base fluid source through the cartridge or around the cartridge via a bypass channel, to vary the concentration of the functional liquid component mixed with the breathable base fluid.

### FIELD OF THE INVENTION

The present invention relates to breathable fluid delivery devices such as electronic cigarettes, and more specifically to a configuration for introduction of chemical species into a breathable fluid stream provided thereby.

### BACKGROUND OF THE INVENTION

In the use of electronic cigarettes, users often prefer an atomized liquid that is flavored and/or combined with physiologically active chemicals, such as nicotine, over one that is not. Prior art achieves the delivery of such a fluid by way of atomizing a mixture of propylene glycol or other suitable chemicals that are mixed with chemical species that provide a flavor or physiological effect, or both simultaneously.

There are several shortcomings of the prior art that utilize this approach. For example, when utilizing a tank for storage of the liquid for atomization, the tank volumes are often relatively large. Users often prefer to fill a tank with a large volume of liquid, which is inconvenient in the sense that a user must either completely consume the liquid or remove the liquid manually and replace it with a different liquid if they desire a different mixture for physiological or flavor reasons. Since it is difficult to completely remove a previous mixture from the components of a tank and atomizing apparatus, the replacement mixtures can interact and create combinations that may be undesirable. A need therefore exists for an atomized liquid delivery system that conveniently allows a user to exchange the flavoring or physiologically active species with ease and without mixing of previously loaded mixtures.

Additionally, prior art utilizing a heating element for atomization of a mixture presents the possibility of degradation and pyrolysis of chemical species residing in the mixture. Therefore, there exists a need for a system whereby the chemical species desired for consumption of the user may not undergo pyrolysis or degradation.

Controlling the temperature of a heating element provides some flexibility in tank chemical composition. However, some chemical components that would be desirable to inhale may be so heat sensitive that they may degrade, conjugate, pyrolize, or otherwise become chemically altered even at the minimum temperatures required to vaporize them. Products of pyrolysis, for example, which may or may not be desirable to inhale, may then be carried by the vapor. Conversely, the greater the fraction of a desirable component that is chemically altered by the heat provided for its vaporization, the lesser the fraction, if any, of the native species that remains unaltered to be carried in the vapor stream. Examples of particularly heat sensitive species include thiosulfinates derived from plants of the Alliacea family. A system is therefore needed that would add more chemical flexibility to tailor a unique experience to each specific user.

### SUMMARY OF THE INVENTION

The present invention relates to breathable fluid delivery systems including an exchangeable fluid permeable cartridge containing a liquid mixture of chemical species to be introduced into a breathable fluid stream passing through the cartridge, which may comprise air in combination with atomized liquid, vapor, or both. The exchangeable fluid permeable cartridge may be disposed in an auxiliary conduit attachment substituting for the mouthpiece of an atomized liquid delivery system (such as an existing electronic cigarette).

In particular, in accordance with an aspect of the present invention, an apparatus for delivering a functional liquid in a breathable fluid stream for inhalation is provided. The apparatus may be an attachment for a breathable fluid delivery device, comprising a mouthpiece, which may be removable, including a distal opening in fluid communication with a proximal opening; and a cartridge comprising a fluid-permeable packing material, configured for drawing a breathable fluid through the packing material by inhaling through the mouthpiece. The packing material contains a functional liquid disposed to be introduced into the fluid stream as the fluid stream passes through the packing material and to be carried in the fluid stream in a breathable form when the fluid stream passes out of the mouthpiece through the distal opening. The functional fluid may provide a flavor, a recreational and/or medicinal drug effect, or other desired effect when the breathable fluid stream is inhaled.

A cartridge suitable for use with embodiments of the invention may consist solely of a fluid permeable packing, or may further comprise additional elements. For example, the cartridge may include a casing covering at least a portion of the packing. Where present, the casing may include open ends or fluid permeable ends that nonetheless provide support to prevent the packing from falling out of or being removed from the casing. Whether the casing includes open ends or fluid-permeable ends that support/retain the packing, the ends of the casing may be sealable for storage or shipping by a removably adhered film. The packing itself may also take different forms, including a single solid porous body, or a mass of fibers, particles, or grains having spaces therebetween, functioning to permit fluid passage, similarly to the pores of a single porous body.

The cartridge may be manually removably retained in a housing, which in turn may be retained supported, and/or enclosed by a housing cover. The cartridge housing includes a proximal opening in fluid communication with the breathable base fluid and a distal opening in fluid communication with the proximal mouthpiece opening, and the housing including structure to support the cartridge in a position in which a portion, such as a proximal end, of the cartridge is in fluid communication with the proximal housing opening and a portion, such as a distal end, of the cartridge is in fluid communication with the distal housing opening. The housing may be removably connected to a fluid delivery device, for example via a coupling adaptor, the fluid delivery device being configured to deliver a stream of the breathable base fluid into the proximal housing opening. Coupling adaptors according to the invention may or may not be interchangeable with existing mouthpieces of existing breathable fluid devices.

When an apparatus according to the invention includes a cartridge housing cover, the housing cover may comprise two separate cover sections removably connected together to at least substantially surround the housing but for a proximal housing cover opening in fluid communication with the proximal housing opening and a distal housing cover opening in fluid communication with the distal housing opening. The two housing cover sections may be threaded together, or, for example, mated together by an insertable portion of one of the sections being slidingly inserted into a receiving portion of the other section. Whether slidingly mated or threaded together, the housing cover sections preferably comprise a resilient member disposed on at least one of the insertable portion and the receiving portion to seal the connection between the housing sections and to frictionally retain the housing sections in a mated configuration.

Preferably, the apparatus or attachment includes a flow channel in which the packing is disposed, the flow channel including a wider portion adjacent at least one of the distal and the proximal end of the cartridge, and a narrower portion adjacent the wider portion and extending therefrom in a direction away from the cartridge. Advantageously, to maximize the surface area available for inflow of breathable fluid through the cartridge, a spacer feature abuts the cartridge to maintain an axial distance between the cartridge and the narrower channel portion. The spacer feature may comprise a separate annular member, or it may be integrated into, for example, a cartridge casing or housing. The spacer may have a uniform longitudinal dimension, or it may have a tapered dimension, so that no part of the proximal/upstream/inlet area of the cartridge is covered so as to block inflow of breathable fluid.

In accordance with another aspect of the invention, a fluid-permeable cartridge for breathable delivery of a functional liquid is provided as a stand-alone product. The cartridge comprises a fluid-permeable packing material containing an absorbed or adsorbed quantity of a functional liquid configured to be introduced into a breathable fluid stream flowing through the cartridge. The cartridge may further include a fluid-impermeable covering disposed over at least a portion of the outer surface area of the packing material. Such a covering may comprise a wall structure generally surrounding the packing material, with or without one or two fluid-permeable ends joined to the wall structure to fully retain/cover/encase the packing. To facilitate shipping and/or extended storage, the covering may include a fluid-impermeable film material removably fixed over the ends of the covering to seal the packing material and functional liquid within the covering for storage and/or transport prior to use of the cartridge.

Suitable film materials may include, without limitation, metal foil and plastics, and the film may be affixed by adhesive and/or heat-fused wax. Packing materials may be selected from among cotton, foam, fibrous media, stacked thread, stone, synthetic porous media, and any other materials having the desired adsorption/absorption and fluid permeability properties.

In accordance with yet another aspect of the invention, a conduit assembly for a breathable fluid delivery device is provided. In particular, a fluid-permeable cartridge is disposed in a main fluid channel for inhalation delivery of a breathable fluid stream, the cartridge containing a functional liquid disposed to be introduced in a breathable form into a breathable base fluid flowing in the main fluid channel through the cartridge. A source of the breathable base fluid is connected in fluid communication with an upstream end of the cartridge—this may be any reservoir of or component or system for delivering a breathable base fluid, including but not limited to a fluid tank and a heating coil or vibration element, for example. A mouthpiece is connected in fluid communication with a downstream end of the cartridge, and an adjustable valve system is incorporated in the assembly for adjusting the flow impedance of a bypass channel passing around the cartridge relative to the flow impedance of a portion of the main channel extending through the cartridge.

In a particular embodiment, the adjustable valve system comprises a tube in fluid communication with the breathable base fluid source, and a tube in fluid communication with the upstream end of the cartridge, one of the tubes being an insertable tube comprising a tapered tip narrowing to a smallest exterior cross section at an open end of the tube, at least a portion of the tapered tip being insertable into an open end of the other tube, the other tube being a receiving tube. The insertable and receiving tubes are relatively movable for insertion and withdrawal of the tapered tip into and out of the receiving tube to vary the cross-sectional area of a clearance between the receiving tube opening and the tapered tip. The bypass channel is in fluid communication with the breathable base fluid source only by way of a passage extending through a clearance between the receiving tube opening and the tapered tip. Preferably, the insertion tube includes a structural feature configured to seal off the receiving tube opening when fully inserted to a closed valve position, to cut off all flow through the bypass channel.

### BRIEF DESCRIPTION OF THE DRAWINGS

FIG. 1 is a transverse plan view of a fluid delivery system attachment in accordance with an aspect of the invention.

FIG. 2 is a transverse cross-sectional view of the attachment shown in FIG. 1.

FIG. 3 is an exploded view of the attachment shown in FIG. 1.

FIG. 4 is an exploded cross sectional view of the attachment shown in FIG. 1.

FIG. 5 is a transverse cross-sectional view of a fluid-permeable cartridge according to another aspect of the invention.

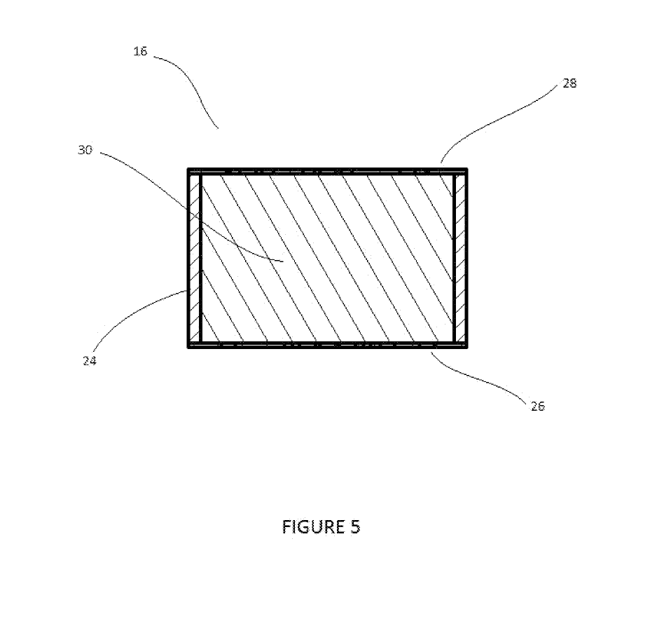

FIG. 6 is an end view of a fluid permeable end of the cartridge shown in FIG. 5

FIG. 7 is an exploded cross-sectional transverse view of the cartridge shown in FIG. 5

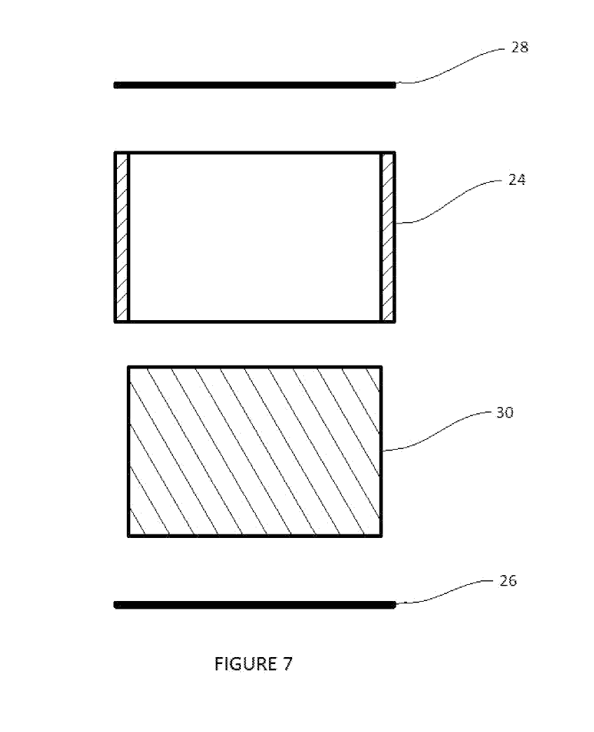

FIG. 8a is a transverse cross-sectional view of another attachment according to the invention, showing a valve in a closed position.

FIG. 8b is a transverse cross-sectional view of the attachment shown in FIG. 8a, showing the valve in a partially open position.

FIG. 8c is a transverse cross-sectional view of the attachment shown in FIG. 8a, showing the valve in a fully open position.

FIG. 9 is a transverse plan view of the attachment shown in FIG. 8a.

FIG. 10 is an exploded perspective view of the attachment shown in FIG. 8a.

FIG. 11 is a transverse cross sectional view of another attachment according to the invention.

FIG. 12a is a transverse plan view of another attachment according to the invention.

FIG. 12b is a transverse cross-sectional view of the attachment shown in FIG. 12a

FIG. 13 is a distal end view of an alternative cartridge housing and an alternative cartridge housing cover section of the attachment shown in FIG. 12a

FIG. 14 is a transverse view of an alternative coupling adaptor component of the attachment shown in FIG. 12a, including an integral valve needle.

FIG. 15a is a proximal end view of a distal cartridge housing cover section of the attachment shown in FIG. 12a.

FIG. 15b is a cross-sectional proximal perspective view of the distal cartridge housing cover section shown in FIG. 15a.

FIG. 16a is a transverse cross-sectional view of an integrated cartridge and valve seat of the attachment shown in FIG. 12a.

FIG. 16b is a transverse cross-sectional view of an alternative integrated cartridge and valve seat for use in the attachment shown in FIG. 12a.

FIG. 16c is an exploded perspective view of the integrated cartridge and valve seat depicted in FIG. 16b.

FIG. 17a is a distal end view of an alternative valve needle and coupling adaptor component accommodating a clicker element.

FIG. 17b is a transverse cross sectional view of the alternative valve needle and coupling adaptor component accommodating a clicker element shown in FIG. 17a.

FIG. 17c is a distal perspective view of the alternative valve needle and coupling adaptor component accommodating a clicker element shown in FIG. 17a.

FIG. 18 is an exploded proximal perspective view of a valve assembly including the alternative valve needle and coupling adaptor component accommodating a clicker element shown in FIG. 17a.

FIG. 19 is a transverse exploded plan view of a complete fluid delivery apparatus incorporating a fluid permeable cartridge and variable valve system in accordance with an aspect of the invention.

### DESCRIPTION OF THE INVENTION

Illustrative embodiments and exemplary applications will now be described with reference to the accompanying drawings to disclose the advantageous teachings of the present invention.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope hereof and additional fields in which the present invention would be of significant utility.

In considering the detailed embodiments of the present invention, it will be observed that the present invention resides primarily in combinations of steps to accomplish various methods or components to form various apparatus and systems. Accordingly, the apparatus and system components and method steps have been represented where appropriate by conventional symbols in the drawings, showing only those specific details that are pertinent to understanding the present invention so as not to obscure the disclosure with details that will be readily apparent to those of ordinary skill in the art having the benefit of the disclosures contained herein.

In this disclosure, relational terms such as first and second, top and bottom, proximal and distal, upper and lower, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises a” does not, without more constraints, preclude the existence of additional identical elements in the process, method, article, or apparatus that comprises the element.

In this disclosure, the use of the term “proximal” with relation to the anatomy of the present invention may be used to distinguish the end of the disclosed assembly that is closest to a coupled fluid delivery device, while the end that would be closest to a user is distinguished as “distal,” without necessarily requiring or implying any actual such relationship or order between such entities or actions.

In this disclosure, the term “fluid” shall be understood to represent a fluid supply of air, preferably carrying with it an atomized, volatilized, nebulized, sprayed/misted, gaseous, or otherwise breathable form of a chemical species that acts to enhance the solvation characteristics of said fluid or acts to deliver energy via a quantity of heat energy, such as through condensation, in either case helping the combined fluid stream to pick up and carry the functional liquid when passing through the cartridge. The supply of air, or of a mixture of air and the aforementioned solvation enhancing or energy delivering species, is referred to herein as the “base breathable fluid,” or “the base breathable fluid stream.” When air alone is used as the base breathable fluid stream, heating the stream will generally help it to pick up and carry the functional fluid as it passes through the cartridge.

For the solvation-enhancing or energy delivering species, existing devices often use propylene glycol, and sometimes glycerin, especially to accommodate users who are allergic to propylene glycol. Transition of the carried chemical species from liquid form to a breathable form is commonly facilitated by rapid heating on a resistive coil or nebulization via a vibrating reed. It is to be appreciated that the present invention is not limited to use with devices utilizing the stated methods and chemicals, but may be utilized with any suitable device capable of transitioning a liquid into a breathable form and making it available for use in the present invention as an airstream providing the desired functions as stated.

In this disclosure, the term “functional liquid” shall be understood to represent a chemical species, or mixture thereof, which is intended to be volatilized, atomized, or otherwise introduced into a fluid stream that is in communication with said liquid. Said liquid may be comprised of any single chemical species or combination of chemical species compatible with substrates upon which they are adsorbed or media into which they are absorbed for use in the present invention.

In accordance with aspects of the present disclosure, breathable fluid delivery device components are presented. In one aspect, the disclosure presents an auxiliary conduit attachment for coupling with electronic cigarettes whereby chemical species can be introduced into a fluid stream provided by a coupled parent electronic cigarette or apparatus capable of forming such a fluid stream as described herein.

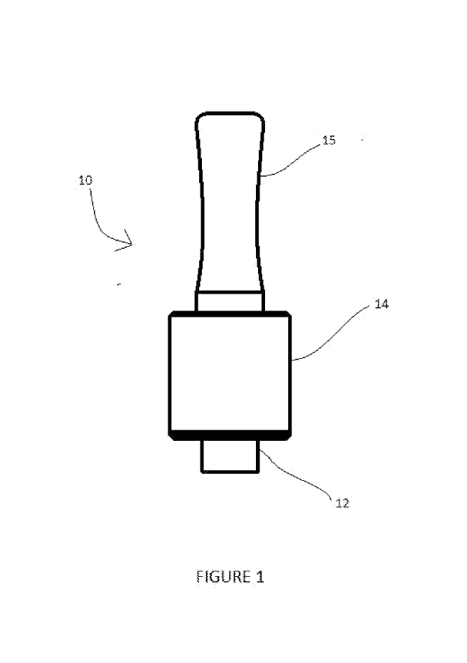

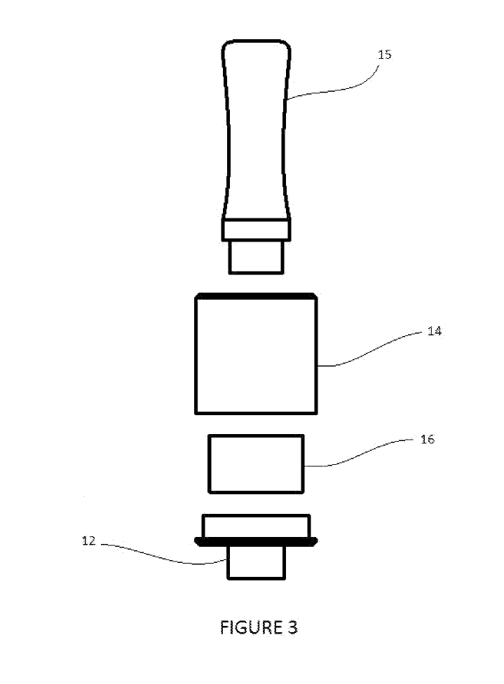

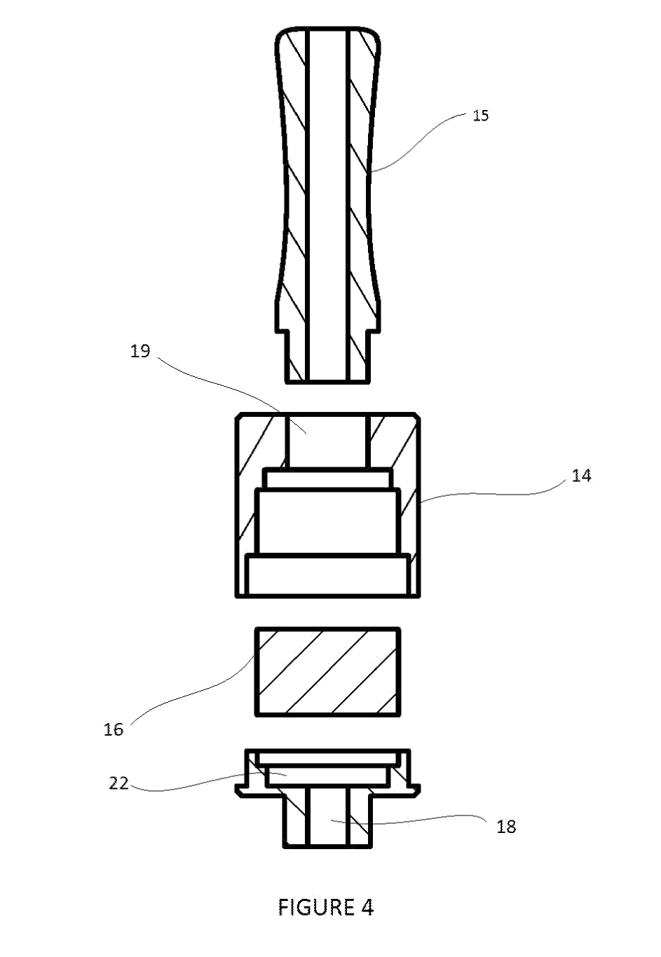

In accordance with a first aspect of the present invention, with reference to an embodiment thereof illustrated in FIGS. 1-4, an auxiliary conduit attachment 10 for coupling with breathable fluid delivery devices such as electronic cigarettes will now be described. Attachment 10 may substitute for a mouthpiece of an existing fluid delivery device, and thus includes a coupling adaptor 12 for attaching to the fluid delivery device as would an existing mouthpiece.

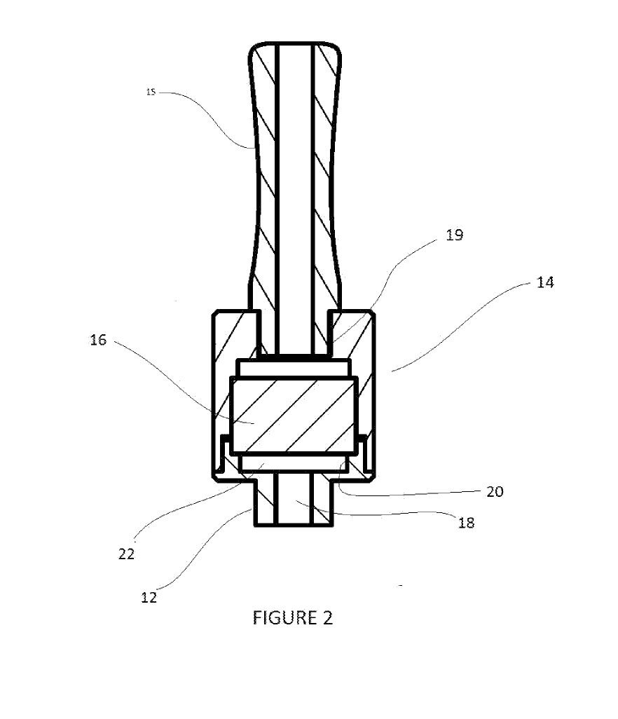

As shown in FIG. 2, a cartridge housing 14 and a coupling adaptor 12 fit together to retain and support between them an exchangeable fluid permeable cartridge 16, capable of containing a functional liquid disposed to be readily picked up and carried in a breathable fluid stream passing therethrough. The proximal end of housing 14 may for example be threaded onto coupling adaptor 12, or attached thereto in any other suitable manner. Housing 14 and adaptor 12 cooperate to provide a defined slot for cartridge 16 to rest in, to prevent cartridge 16 from easily sliding or falling out of housing 14 when being exchanged by a user. Additionally, the housing slot does not severely obstruct access to cartridge 16, so as to make it relatively easy for a user to exchange cartridge 16 by grasping it with one's fingers.

To permit the passage of a breathable fluid through attachment 10, coupling adaptor 12 includes an open channel 18 spanning its internal length, which may or may not have a similar inner diameter to that of a mouthpiece for which attachment 10 substitutes, and housing 14 also includes a channel for receiving a mouthpiece 15. In a preferred embodiment, the housing cover includes a distal end wall with a channel 19 passing through it designed to accept mouthpiece 15. Alternatively, the housing cover may comprise an integrated mouthpiece (not shown). Various embodiments could adapt to various mouthpieces. In the illustrated embodiment, mouthpiece 15 has the same dimensions as coupling adaptor 12. In other various embodiments, the dimension of the coupler and the dimension of the channel could vary and do not necessarily need to be compatible in terms of mouthpiece attachment male and female fittings.

Attachment 10 may be constructed with various fluid delivery device coupling adaptors to permit use of attachment 10 with said devices at a connection point where a mouthpiece of an existing type would normally fit, although coupling adaptor 12 may alternatively comprise a different type of connector

The proximal end of the slot for cartridge 16 includes a structural spacer feature, illustrated in FIG. 2 as a stepped profile 20 of coupling adaptor 12. The spacer feature acts to lift the exchangeable cartridge off of the proximal base of the housing slot where a fluid enters from an opening, illustrated as the distal end of channel 18, having a cross-sectional area smaller than the area of an volume 22 defined by the riser of the stepped profile of coupling adaptor 12. Thus, the spacer feature functions to increase the cross sectional area where a breathable fluid stream may enter cartridge

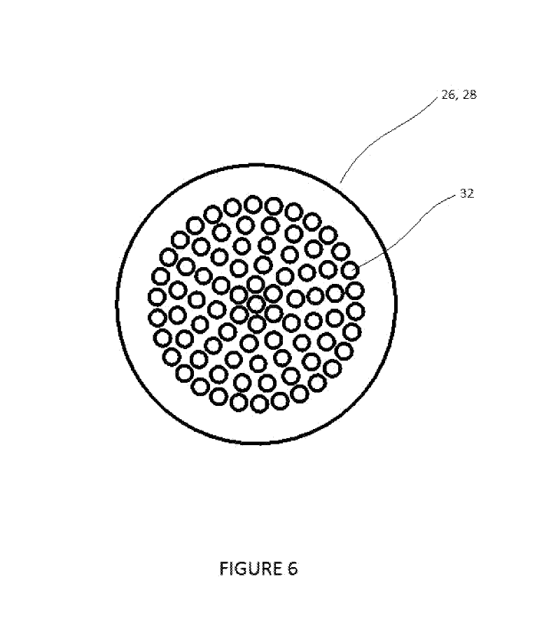

Cartridge 16 may comprise a casing 24, fluid permeable ends 26 and 28, and a fluid permeable packing 30, which may for example be a single porous body or a mass of fibers, coarse grains, or particles of material including spaces therebetween, even if the individual fibers, grains, or particles are themselves non-porous. Casing 24 is preferably a tube-like structure having a hollow channel spanning its length and open ends. At the ends of casing 24, fluid permeable ends 26 and 28 are connected or attached to allow the passage of a fluid through the casing channel and packing 30, while providing containment of packing 30 inside casing 24. Fluid permeable ends 26 and 28 may be, but are not limited to, mesh screens or perforated sheets having arrayed apertures 32, as illustrated in FIG. 6. As an additional aspect, one or both of ends 26 and 28 may be constructed from the same piece of material as the casing 24, but with fluid permeable properties. Ends 26 and 28 may be constructed to permit sealing with a thin film or the like (not shown) for storage and packaging of cartridge 16. This film may for example be metal foil, plastic, or other comparable materials that could be fixed to ends 26 and 28 via an adhesive, heat fused wax, or other comparable method.

Packing 30 may be composed of any suitable material that is fluid permeable and does not pose an inhalation health risk. Suitable materials for packing 30 include cotton, foam, stacked thread, porous stone, synthetic porous media, or any other material which is capable of adsorbing or absorbing the desired chemical species in liquid phase. Packing 30 is configured to accept a functional liquid, while still maintaining fluid permeable properties. In particular, packing 30 should be capable of holding a reasonable quantity of the functional liquid before becoming saturated, to avoid the need for frequent replacement of cartridge 16.

In certain preferred pairings of a packing material of packing 30 with a functional liquid, the surface chemistry of the packing material favors the adsorption of the functional liquid in order to improve saturation and functional liquid load. For example, a porous media having a surface chemistry that is hydrophilic may better saturate with hydrophilic functional liquids. A sintered porous plastic has proven to be a particularly effective porous medium, due to its tendency to force an air flow to spread generally evenly across its entire cross sectional area, thereby exposing a greater volume of air to the functional liquid. This widening/dispersion of the air stream is believed to be largely due to a significant flow resistance produced by the material, as evidenced by a noticeable pressure drop across the length of cartridge 16 when packed and saturated. Other media may exhibit similar flow properties with similar effects.

Functional liquids that may be advantageously contained in cartridges according to the invention include, without limitation, esters, acetate esters, alcohols, acids, lactones, carbonyls, terpenes, thiols, saturated and unsaturated thiosulfinates, hemiterpenes, monoterpenes, sesquiterpenes, diterpenes, sesterterpenes, triterpenes, sesquiterpenes, tetraterpenes, polyterpenes, norisoprenoids, and derivatives thereof, such as terpin hydrate, a derivative of turpentine; natural flavor compounds such as those often found in fruits, including but not limited to: Gamma Decalactone, Gamma Octalactone, Butyric Acid, 2-Methyl Butyric Acid, Proprionic Acid, Isovaleric Acid, Isobutyric Acid, Cinnamic Acid, Phenethyl Alcohol, Ethyl Butyrate, Ethyl Isobutyrate, Ethyl-2-Methyl Butyrate, Ethyl Isovalerate, Methyl Cinnamate, Ethyl Proprionate, Ethyl Hexanoate, Isoamyl Isovalerate, Phenethyl Acetate, (Z)-3-hexenal, beta-ionone, hexanal, beta-damascenone, 1-penten-3-one, 3-methylbutanal, (E)-2-hexanal, 2-isobutylthiazole, 1-nitrophenylethane, (E)-2-heptenal, furanones, 2,5-dimethyl-4-hydroxy-3(2H)-furanone, methyl 2-methylbutanoate, ethyl 2-methylpropanoate, methyl hexanoate, methyl butanoate, trans-2-hexenal, ethyle-2-methylbutanoate, ethyl butanoate, trans-2-hexenol, hexyl acetate, hexyl butanoate, 1-butanol, 1-hexanol, cis-3-hexenal, cis-3-hexeol, cis-3-hexenyl acetate, ethyl hexanoate, propyl 2-methylbutanoate, 2-methyl-1-butanol, benzyl alcohol, 1-octanol,2-phenylethanol, 1,3-oct-5(Z)-enediol, 1,3-octanediol, 4-vinylguaiacol, eugenol, 2-methylbutanoic acid, 4-hydroxyphenylacetic acid, 3-hydroxy-beta-damascone, 4hydroxy-3-methoxyphenylacetic acid, 3-oxo-alpha-ionol, vomifoliol, 3-Oxo-β-ionol, dehydrovomifoliol, roseoside; and/or natural flavor compounds such as those found in vegetables, including but not limited to: dimethyl sulfide, thiosulfinates, disulfides, poly-sulfides, 2-propene-1-sulfinothioic acid S-2-propenyl ester (allicin), methanesulfinothioic acid S-2-propenyl ester, 2-propene-1-sulfinothioic acid S-(E,Z)-1-propenyl ester, 2-propene-1-sulfinothioic acid S-methyl ester, Linoleic acid, (E)-2-nonenol, (E)-2-nonenal, (Z)-3-nonenol, (Z)-3-nonenal, C9 Carbonyls, (Z,Z)-3,6-nonadienal, (E,Z)-2,6-nonadienal, 3-methylbutanoates, 2-phenethyl esters, 2-phenethyl 3-methylbutanoate, (E)-2-hexenyl 3-methylbutanoate, benzyle 3-methylbutanoate, (E)-2-hexenyl 3-methylbutanoate, benzyl 3-methylbutanoate, methyl 3-methylbutanoate, butyl 3-methylbutanoate, 3-methylbutanoate, butyl 3-methylbutanoate, 3-methylbutyl 3 methylbutanoate, (E)-2-pentenyl 3-methylbutanoate, 2-phenethyl hexanoate, sesqunterpene alcohol, cubenol, phthalides, 3-butylphthalides, 3-butuyl-4,5-dihydrophalide, cis and trans forms of 3-butyl-3a,4,5,6-tetrahydrophthalide, (Z)-ligustilide, 1-(E,Z)-3,5,-undecatriene, sesquinterpene hydrocarbons, alpha-copane, alpha-muurolene, alpha-calacorene, cadinenes, 2-acetyl-1-pyrroline, 2-ethyl-3,6-dimethylpyrazine, acetaldehyde, 3-methylbutanal, 4-vinylguaiacol, 2-acetylthiazole, 2-acetyl-2-thiazoline, 2-(1-hydroxyehtyl)-4,5-dihydrothiazole, 2,5-Dimethyl-4-hydroxy-3(2H)-furanone, hydrogen sulfide, methanethiol, ethanethiol, octa-1,5-dien-3-one, linolool, (E,E)-deca-2,4-dienal, p-mentha-1,3,4-triene, myrcene, 2-sec-butyl-3-methoxypyrazine, myristicin, (E, E)-deca-2,4-dienal, (Z)-dec-6-enal, Beta-phellandrene, (Z)-hex-3-enal, (Z)-hex-3-enol, (Z)-hex-3-enyl acetate, vanillin, menthol, methyl salicylate, 3,7-guaiadiene, delta-cadinene, cannabinoids, nicotine, caffeine, citicolene, and taurine. The current invention may also employ the vast array of melanoidins, a class of chemicals produced by Maillard reactions, wherein amino acids and reducing sugars are heated together to produce complex compositions of chemicals derived therefrom. In addition, extracts from plants and other biological materials may be utilized. Pharmaceutical inhalation delivery drugs may also be utilized, such as Ciclesonide, Cromolyn Sodium, Ipratropium Bromide, Nedocromil Inhalation, Albuterol Sulfate, Triamcinolone Acetonide, Albuterol Sulfate, Levalbuterol Tartrate, Flunisolide Hemihydrate, Fluticasone Propionate, Salmeterol, Fluticasone Propionate, Paclitaxel, Salmeterol Xinafoate, Metaproterenol Sulfate, Beclomethasone Dipropionate HFA, Beclomethasone Dipropionate Monohydrate, Ribavirin, N-acetyl-L-cysteine, Loxapine, Insulin, Pirbuterol, Budesonide, Formoterol Fumarate Dihydrate, Methacholine Chloride, Mometasone Furoate, Pentamidine Isethionate, Dornase alfa, Iloprost, Tobramycin, Fluticasone Propionate, Arformoterol Tartrate, Idarubicin, Levalbuterol.

As an alternative to cartridge 16 as illustrated, a cartridge within the scope of the invention may consist of only an open ended casing and a fluid permeable packing material residing inside the casing, such as a porous body, a quantity of particulate material, or a mass of fibers. The embodiment may contain the packing partially or completely saturated with a said functional fluid and the cartridge as a whole may be exchangeable in the same fashion as that of the disclosed embodiment consisting of casing 24, packing 30, and fluid permeable ends 26, 28.

In another embodiment, the cartridge may consist solely of a packing material, which may not necessarily have an outer casing or fluid permeable ends. The porous material may be partially or completely saturated with a functional fluid and may be exchangeable in the same fashion as that of the disclosed embodiment having casing 24 and fluid permeable ends 26, 28.

Individual parts of attachments or fluid delivery devices according to the invention may be constructed out of any suitable material that permits ease of use thereof, durability, safety, and ease of manufacturing. In preferred embodiments, components are generally composed of a relatively hard, durable, and non-corrosive material, such as stainless steel, aluminum, brass, graphite, ceramics, silicon carbide, certain plastics or other suitable materials. Plastics used for components of an inhalation device according to the invention should generally be highly chemically resistant, as some functional fluids, such as certain alcohols, have been shown to cause degradation of certain existing plastic mouthpieces and polycarbonate parts. Suitable plastics may include silicones, thermoplastic elastomers/TPEs, Santoprene®, polytetrafluoroethylene (PTFE), polyaryletherketone family plastics, such as PEEK (polyether ether ketone), PVDF (polyvinylidene difluoride), PVC (polyvinyl chloride), CPVC (chlorinated polyvinyl chloride), Nylon®, Teflon®, HDPE (high density polyethylene), LDPE (low density polyethylene), Acetal, ABS (Acrylonitrile butadiene styrene), Halar®, Fluorosint®, Polypropylene, Polysulfone, PPS (polyphenylene sulfide), Torlon®, UHMW (ultra-high-molecular-weight polyethylene), CAB (cellulose acetate butyrate), Ertalyte®, Nylatron®, Acetron®, TIVAR®, Proteus®, and Sanalite®.

In preferred embodiments, materials used do not pose a significant health risk to users under normal use conditions, and should be selected to be compatible with the functional fluids used. For example, some functional fluids suitable for use according to the invention, including alcohols and terpenes, are corrosive to certain materials that are otherwise desirable for use in the devices of the invention. Polycarbonate is an example of a clear, hard plastic that may be advantageously used for device components, so long as it is not exposed to certain functional fluids that may damage it. Coupling adaptor 12 may be coated in a protective coating such as a paint, powder coating, film coating, electroplated coating, or any other suitable coating compatible with the material of construction.

The foregoing description is provided to enable any person skilled in the relevant art to practice the various embodiments described herein. Various modifications to these embodiments will be readily apparent to those skilled in the relevant art, and generic principles defined herein can be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the relevant art are expressly incorporated herein by reference and intended to be encompassed by the claims. Nothing disclosed herein is intended to be dedicated to the public.

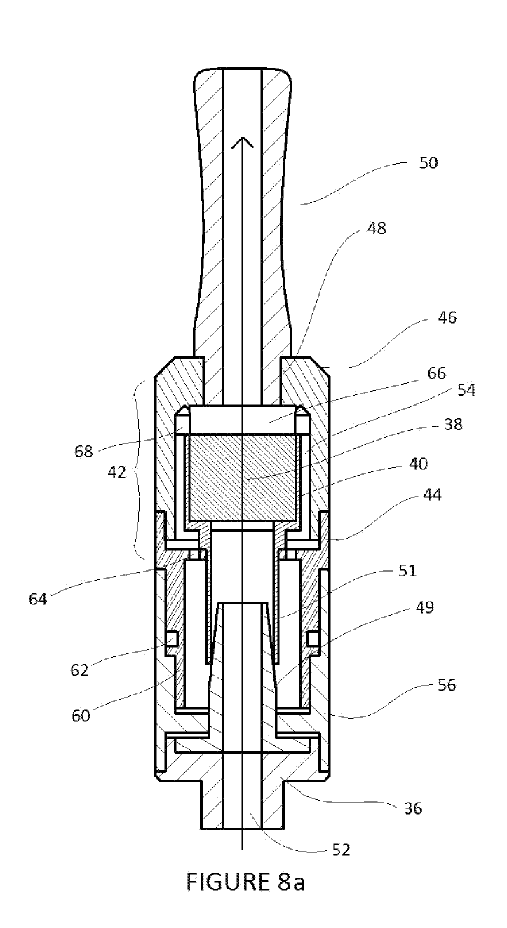

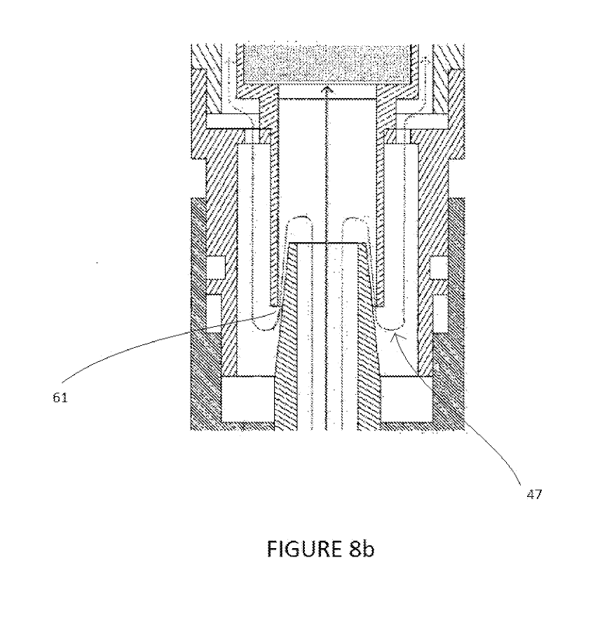

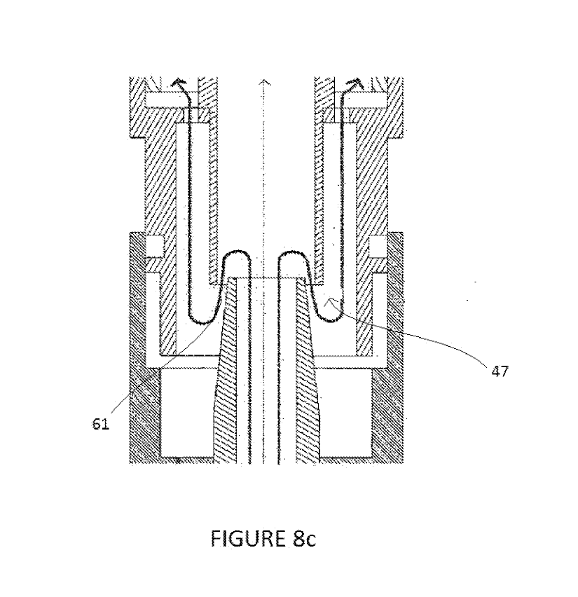

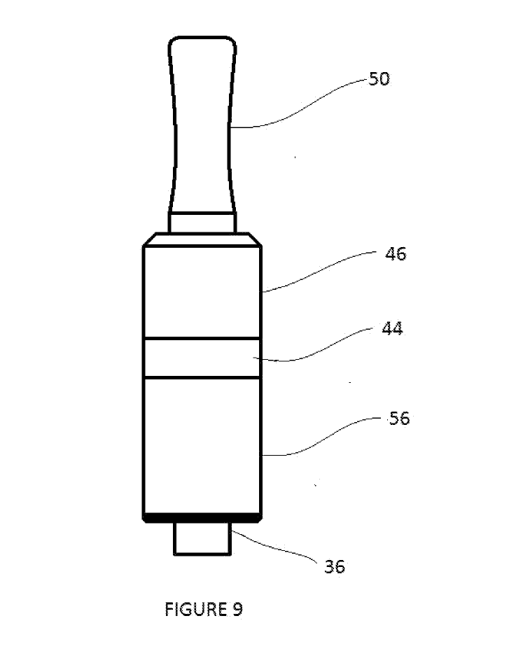

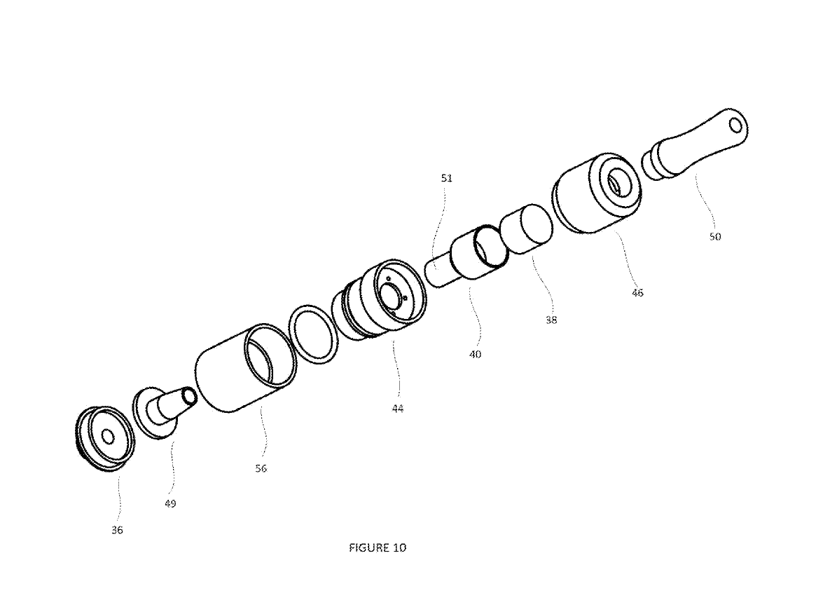

In accordance with a second aspect of the present invention, with reference to a first embodiment thereof illustrated in FIGS. 8-10, a variable flow attachment 34 is provided for coupling with breathable fluid delivery devices such as electronic cigarettes. Attachment 34 comprises a coupling adaptor 36 for attachment to the fluid delivery device (not shown), a variable valve 47 comprising a valve needle 49 cooperating with a valve seat 51 to throttle the portion of fluid passing through or around an exchangeable fluid permeable cartridge 38, a cartridge housing 40 for retaining and supporting cartridge 38, and a housing cover 42 comprising mating sections 44 and 46 for locking a fluid permeable cartridge into housing 40, having also a channel 48 for receiving a mouthpiece 50. Cartridge housing 40 is illustrated as integrally incorporating valve seat 51.

Attachment 34 may be constructed with various fluid delivery device coupling adaptors to permit use of attachment 34 with said devices at a connection point where a mouthpiece of existing such devices would normally be fitted, although not exclusively limited to such existing connection types. Thus, in the illustrated embodiment, coupling adaptor includes the same type of connection as mouthpiece 50. To permit the passage of a fluid through attachment 34, coupling adaptor 36 includes an open channel 52 spanning its internal length. The inner diameter of channel 52 is optionally but preferably similar to that of a mouthpiece for which the herein described auxiliary conduit assembly is substituted.

Attachment 34 may take on various embodiments. Preferably, the valve system is able to partition a fluid flow either through or around cartridge 38, allowing a user to select what portion of the total fluid flow is exposed to the functional fluid and what portion is not, to vary the intensity of the flavor or other functional effect in the inhaled stream. In the illustrated embodiment, a maximum intensity is delivered by fully seating valve needle 49 into valve seat 51 to close a bypass channel 54, and a minimum intensity is delivered by withdrawing valve needle 49 to the farthest extent permitted by the device construction to provide the freest access to bypass channel 54. The illustrated valve is adjusted by twisting a threaded connection between proximal housing cover section 44 and a valve housing 56. Such a threaded connection is a convenient way to provide fine adjustment control, but other valve adjustment mechanisms, including relatively sliding components, may be alternatively provided.

In other embodiments not shown, it may also be possible for a user to fully close the main channel that passes through the cartridge to reduce the intensity of the functional effect to essentially zero. For example, such a feature could be used to produce a “chaser” effect, if the functional liquid has an unpleasant taste, by a user quickly following a breath containing the functional fluid with a breath containing the base breathable fluid only, which may contain an agent that desirably masks or alters the flavor of the functional fluid. This throttling of fluid flow between the two flow paths would preferably be achieved by a valve system between the fluid entering through the open ended channel in the said fluid delivery device coupling adaptor and a housing associated with the coupling adaptor that is proximal to the valve.

In still other embodiments not shown, a plurality of cartridges may be arranged in a device, either in parallel or in series, being disposed in one or more fluid flow pathways leading to one or more mouthpieces. Valve systems of such a device may include one or more valves similar to that of the illustrated embodiment, such as one valve for each cartridge, for example. The valve or valves may simply partition the inhaled fluid stream through or around a single one of the cartridges, or may variably distribute the stream between or among two or more of the cartridges, for variable selection of flavors, functions, or combinations thereof.

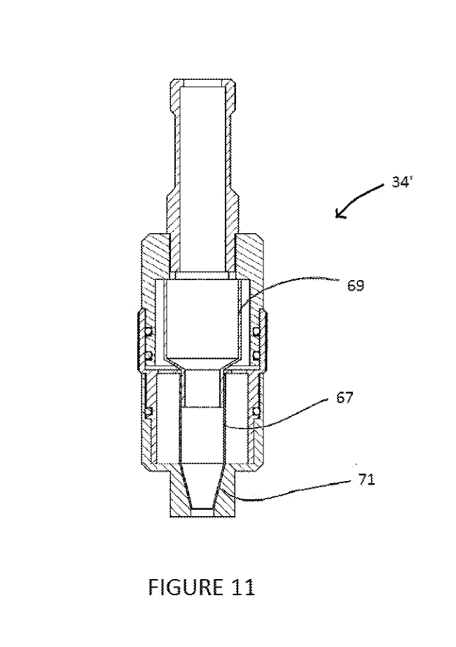

The illustrated valve is somewhat similar to a needle valve, but with valve needle 49 including an open ended channel 58 spanning its interior length. Interacting with valve needle 49 is valve seat 51, which is illustrated as a tube into and out of which valve needle 49 can be drawn. In the embodiment illustrated in FIGS. 8-10, valve needle 49 is positioned adjacent coupling adaptor 36 and positioned upstream of valve seat 51, while valve seat 51 is integral to cartridge housing 40. This arrangement could also be reversed, generally as in the embodiment illustrated in FIG. 11 as attachment 34′, including a valve needle 67 associated with a cartridge housing 69 and configured to insert in the proximal direction into a valve seat 71 associated with a coupling adaptor, or in the illustrated case, integrally formed in a coupling adaptor. However, the upstream/proximal position of valve needle 49 is believed to be slightly preferable for multiple reasons. For instance, baffling the bypass flow pathway to double back on itself, as illustrated by the arrows provides additional flow resistance and thus a finer adjustment of the bypass flow for a given amount of twist of the threaded valve assembly. Also, the doubled back bypass flow arrangement makes the parts easier to machine and injection mold, as well as improving the ease of handling of the fluid permeable cartridges, since they are larger and easier to manipulate with one's fingers. A long cartridge tube also makes it easy to load and unload the cartridge.

An exchangeable cartridge porous packing, containing a functional fluid, impedes the flow of fluid through it to some extent. This flow impedance may be significant without departing from the scope of the invention, but it should not completely block the flow of fluid through the packing when a pressure differential is applied across its length. With respect to the measure of the pressure differential across the packing, in the preferred embodiment the pressure differential considered for intended function would correspond to a vacuum which could be comfortably provided by the intended user drawing in a breath through the device in normal atmospheric conditions. In certain variations and embodiments which could be envisioned by someone skilled in the art provided in this disclosure, the pressure differential measurement may be a value dependent upon other parameters and it is to be understood that various pressure differentials could be considered for design of the flow impedance of the packing to permit intended function of the variable valve system.

Under a pressure differential across the combined length of valve housing 56 and cartridge housing 42, variable valve 47 facilitates the partitioning of fluid flow between the two flow paths in relation to the flow impedance of the cartridge packing and the cross sectional area of a gap 61 that is present between valve needle 49 and valve seat 51 in partially open and fully open valve positions, as illustrated in FIGS. 8b and 8c. As a further aspect of variable flow attachments according to the invention, a structural relationship is provided to allow a user to draw the valve seat away from or onto the valve needle. As illustrated in FIG. 8, this is embodied as a movable connection between proximal cartridge housing cover section 44 and valve housing 56, which may include a threaded connection between valve housing 56 and a proximal threaded end 60 of section 44. Preferably the gap between the section 44 and valve housing 56 is sealed from fluid flow such as air leaks, by an O-ring or analogous suitable sealing structure mounted in a gasket seat 62 of proximal cartridge housing cover section 44. Preferably, the O-ring or substituted structure would consist of a resilient material or any other suitable material that would be chemically compatible with the chemicals being passed through the channel between the needle valve, housing, and sleeve.

With reference to FIG. 8, valve seat 51 is docked in a collar portion of section 44, the collar portion including bypass channel inlet apertures 64 for permitting the free flow of fluid diverted around cartridge 38.

Cartridge housing 40 cooperates with the interior of housing cover 42 to define bypass channel 54 to permit the flow of fluid around cartridge 38 to mouthpiece 50. More specifically, the cartridge housing has an exterior diameter less than that of the interior diameter of the housing cover. Mixing of the bypass fluid stream with the fluid stream that passes through cartridge 38 occurs in a widened channel area 66 in fluid connection with the distal end of cartridge 38 and with bypass channel 54 via a reentry passage 68 formed in a portion of distal cartridge housing cover section 46.

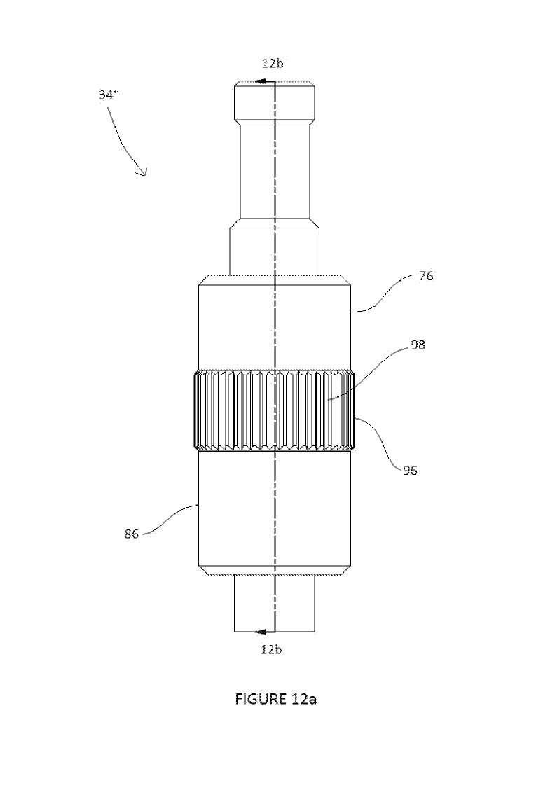

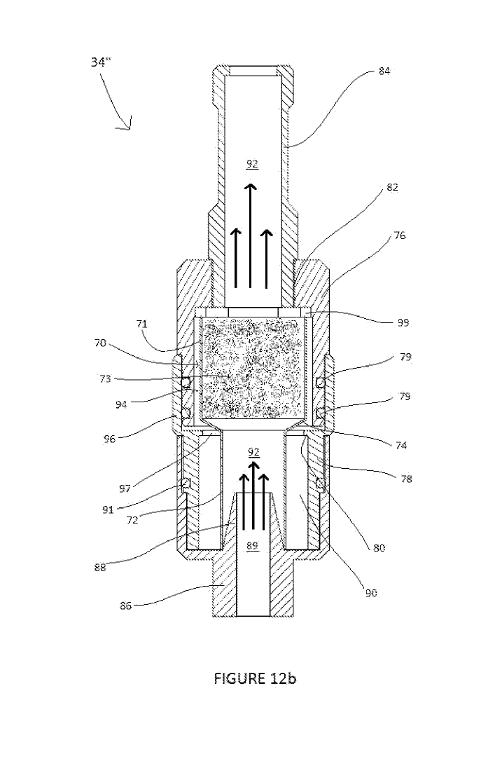

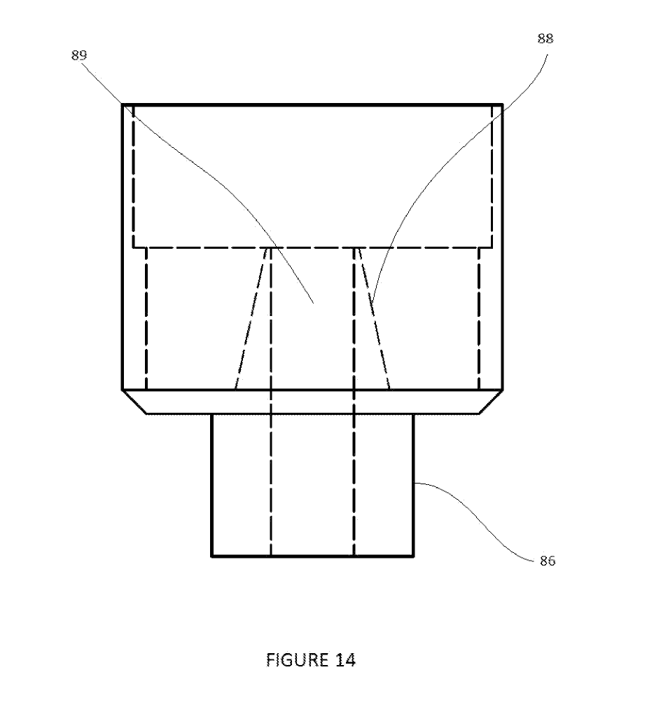

In another embodiment of a variable-flow attachment according to the invention, illustrated in FIGS. 12a-16, an attachment 34″ that is functionally similar to attachment 34 is illustrated, including an improved valve adjustment feature. Attachment 34″ includes a cartridge 70 including an integral casing 71 with an integrally attached, tubular valve seat 72, having a smaller bore diameter, extending proximally therefrom. Instead of a perpendicularly stepped profile as illustrated for cartridge housing 40 of attachment 34 described above, cartridge casing 71 includes an oblique tapered section 74 transitioning between its wider and narrower channels, so that essentially no portion of the proximal face of a circular cylindrical packing 73 inserted contained therein is blocked to flow of the breathable fluid in the distal direction.

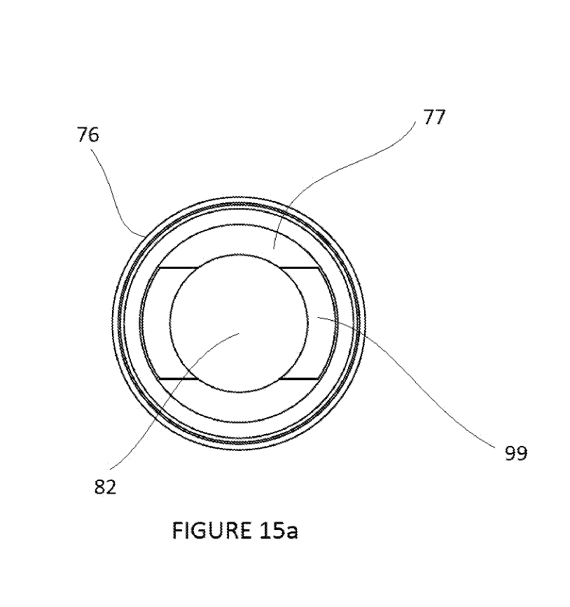

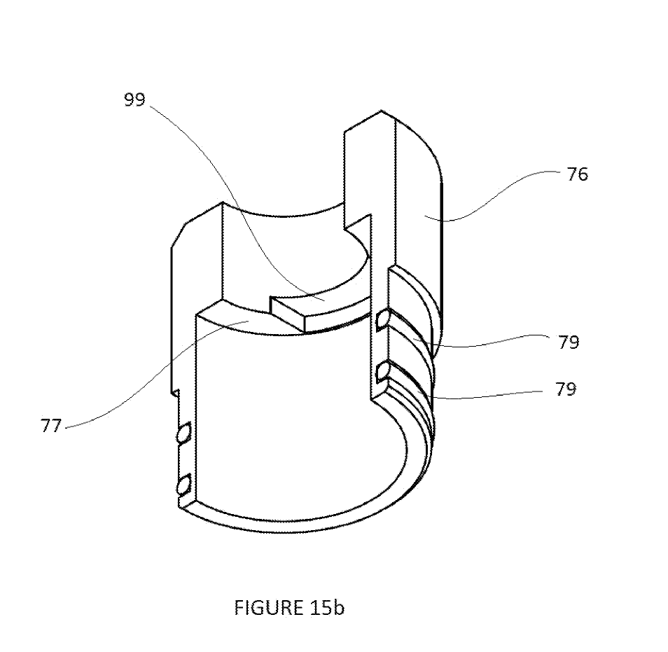

A distal cartridge casing cover section 76 and a proximal cartridge casing cover section 78 cooperate to enclose and retain cartridge casing 71, section 76 being slidingly inserted into section 78. The sliding connection between sections 76 and 78 is preferably sealed by O-rings 79, depicted in FIG. 12b as mounted in annular O-ring seat channels formed in the exterior of the inserted portion of section 76. In its retained position, a distal end of cartridge 70 abuts a proximally facing annular interior end face 77 (seen in FIGS. 15a and 15b) of section 76, and the generally proximally facing exterior surface of tapered section 74 abuts a collar 80 of section 78, essentially to prevent movement of cartridge housing 70 in either axial/longitudinal direction with respect to attachment 34″. Section 76 includes a distal channel 82 for carrying breathable fluid flow exiting from a cartridge 70 and for receiving a mouthpiece 84 inserted into its distal end. Section 78 is threaded into a coupling adaptor 86 which also includes an integral valve needle 88 having a channel 89 extending therethrough for permitting breathable fluid to pass from a delivery apparatus connected to coupling adaptor 86 into the interior of valve seat 72. The connection between section 78 and coupling adaptor 86 is sealed by an O-ring 91.

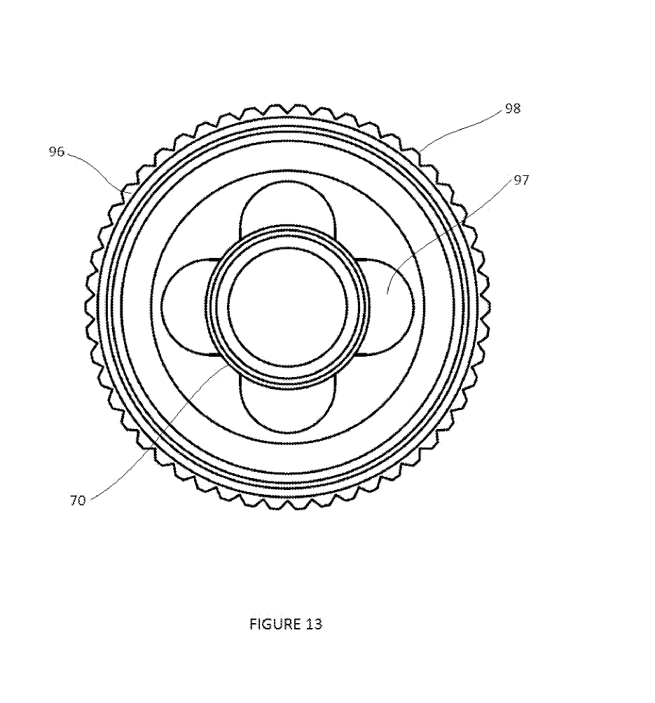

A variable valve 90 comprising valve seat 72 and valve needle 88 serves to variably proportion the flow of a breathable fluid stream entering through coupling adaptor 86 between a main channel 92 extending through a cartridge in cartridge housing 70 and a bypass channel 94 passing around cartridge 70. Similarly to the function of valve 47 of attachment 34, breathable fluid is permitted to pass through variable valve 90 into bypass channel 94 only in a proportion permitted by a clearance (if any) between valve seat 72 and valve needle 88 is opened by withdrawing valve seat 72 from valve needle 88. Valve 90 is depicted in FIG. 12b in a fully closed position with no clearance between valve needle 88 and valve seat 72, and thus all of the breathable fluid stream is constrained to pass through a cartridge (not shown) retained in cartridge housing 70. When valve 90 is opened, breathable fluid is permitted to pass through a clearance between valve needle 88 and valve set 72, through bypass channel inlet apertures 97 formed in collar 80 (shown in FIGS. 12b and 13) into bypass channel 94, and through a reentry passage 99 formed in distal cartridge housing cover section 76 (shown in FIGS. 15a and 15b) to re-enter main flow channel 92.

Valve 90 is opened by a user unscrewing proximal cartridge casing cover section 78 from coupling adaptor 86 section 78 with a twisting motion facilitated by a rotatable ring portion 96 of section 78, having a grip enhancing textured outer surface 98 with an exterior diameter larger than that of the adjacent exterior surfaces of distal cartridge casing cover section 76 and coupling adaptor 86.

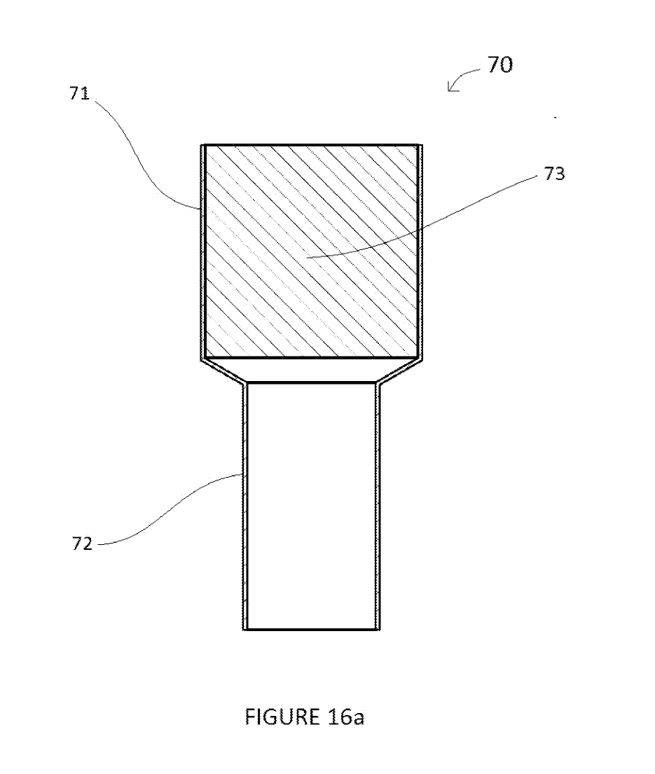

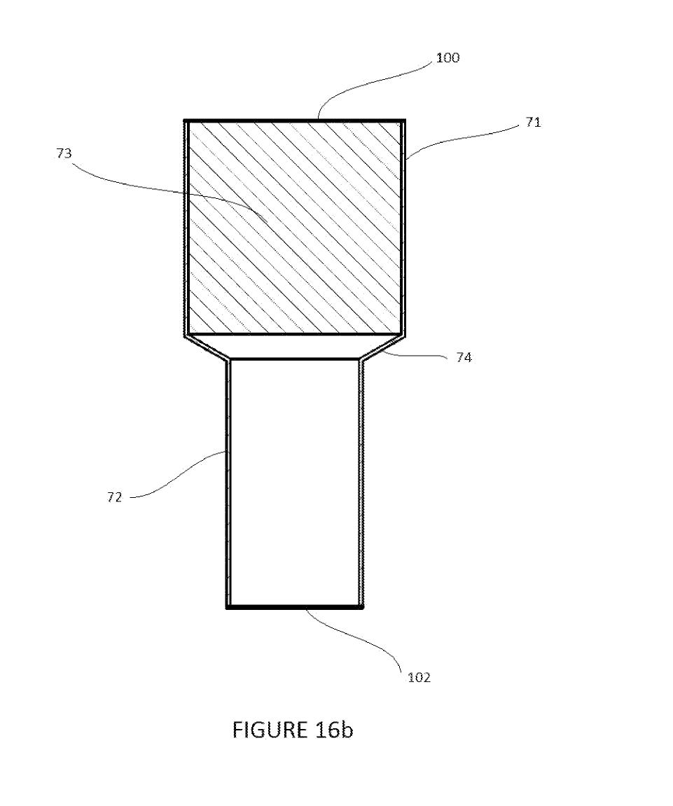

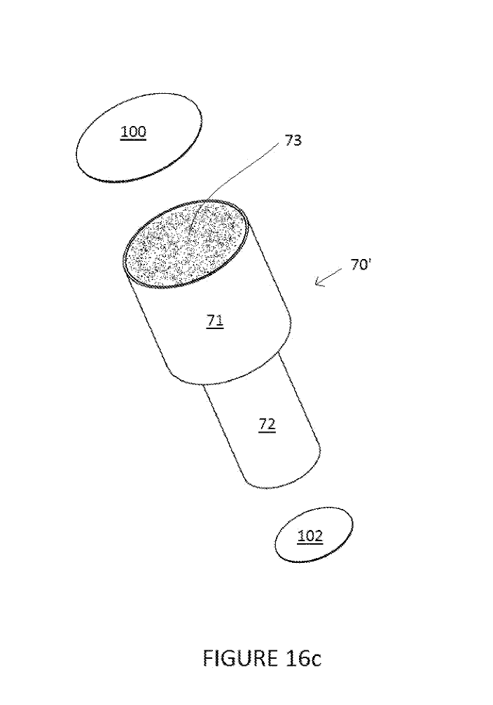

Turning to FIGS. 16a-16c, cartridge 70 of attachment 34″, and an alternative cartridge 70′, are illustrated in detail. In FIG. 16a, cartridge 70 is shown to consist only of casing 71 and packing 73. On the other hand, an alternative cartridge 70′, as depicted in FIGS. 16b and 16c, may include a distal fluid permeable end cover and/or sealant film 100, and optionally a proximal sealant film 102. Sealant film would serve the purpose of sealing functional liquid inside cartridge 70′ for shipping, handling, and/or storage, and any sealant film would be removed prior to use of cartridge 70′ in attachment 34″.

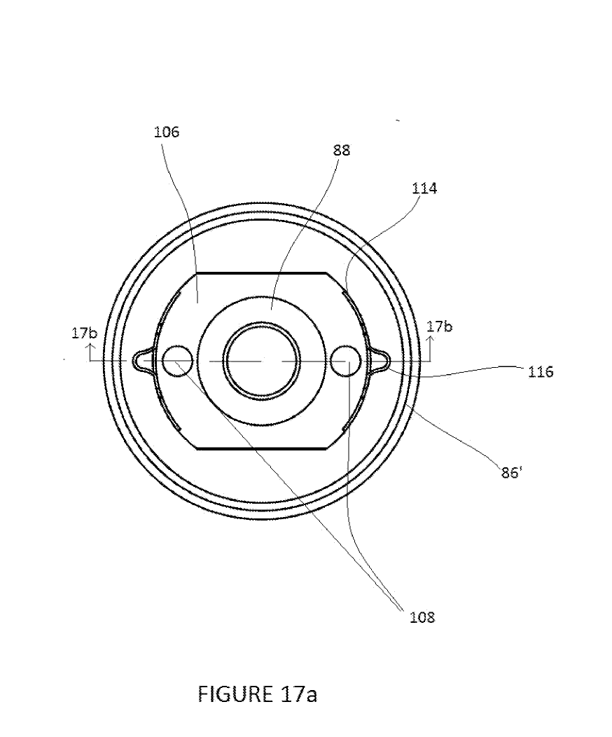

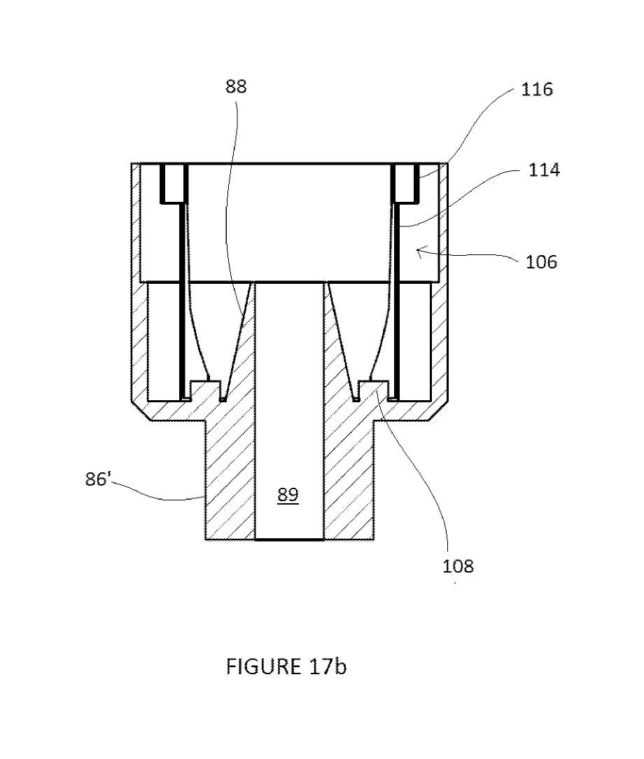

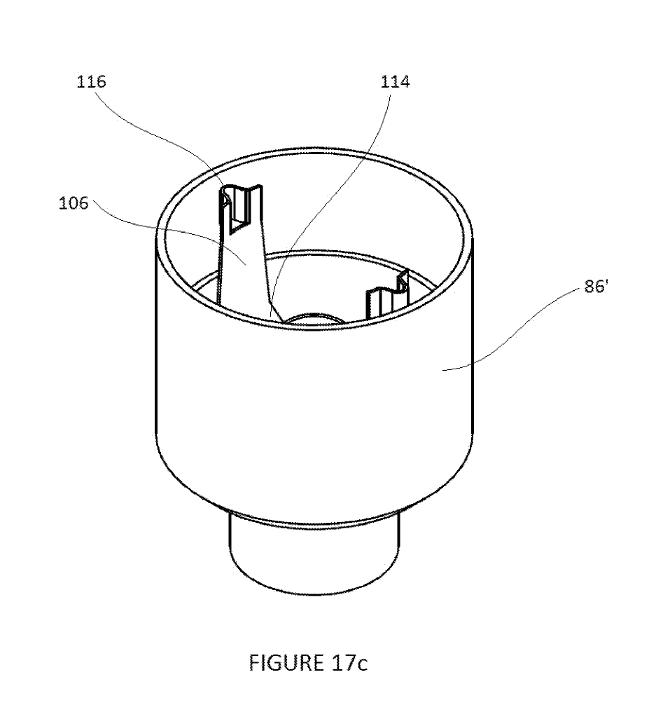

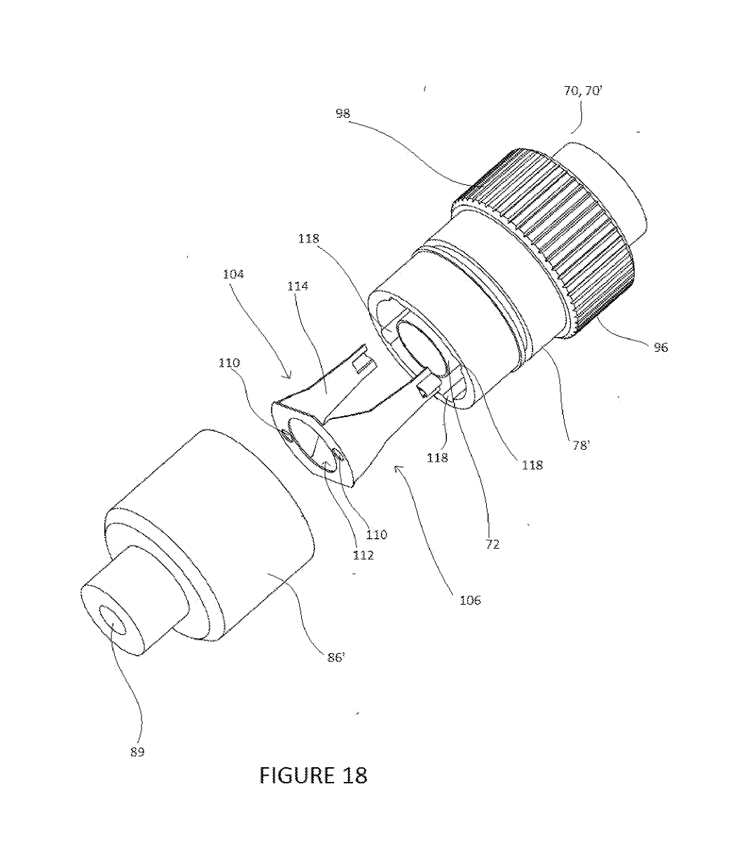

Referring to FIGS. 17 and 18, an alternative coupling adaptor and valve assembly 104 (full assembly shown in FIG. for use in attachments similar to attachments 34, 34′, and 34″ is depicted as including a “clicking” mechanism, providing sensory feedback to a user to facilitate returning the valve assembly to a variable valve position that the user finds to his or her liking. In particular, valve assembly 104 includes a clicker element 106 accommodated between an alternative valve needle component/coupling adaptor 86′ and an alternative proximal cartridge casing cover section 78′ by features added thereto with respect to the illustrated coupling adaptor 86 and section 78 described and illustrated above. In particular, clicker element 106 is retained in and constrained to rotate with valve needle component 86′ by a mating connection between posts 108 of valve needle component 86′ and holes 110 in a proximal base of clicker element 106, a central hole 112 in clicker element 106 at the same time accommodating valve needle 88 extending therethrough. Clicker element 106 further includes flexible arms 114 having radial clicking protrusions 116 at their distal ends. For insertion of section 78′ into valve needle component 86′, clicking protrusions 116 are aligned with and inserted into longitudinal slots 118 formed in the interior of section 78′. In the illustrated embodiment, there are four longitudinal slots 118 and two clicking protrusions 116; however, there may be as many longitudinal slots 118 as desired, or as few as one, although it is preferred that there be at least as many slots as protrusions, including protrusions arranged for simultaneously longitudinally receiving each protrusion. It is also preferred that slots 118 be evenly spaced apart, so that a number of clicks may provide a user with a tactile and auditory indicator of an amount of twisting corresponding to a desired variable valve position. When the threads (not shown) of section 78′ engage those of needle valve component 86′ and a user begins to twist the two together, a tactilely pleasing and informative clicking sound and sensation will occur each time a clicking protrusion snaps into one of slots 118, corresponding in the illustrated embodiment to a quarter turn of rotatable ring portion 96.

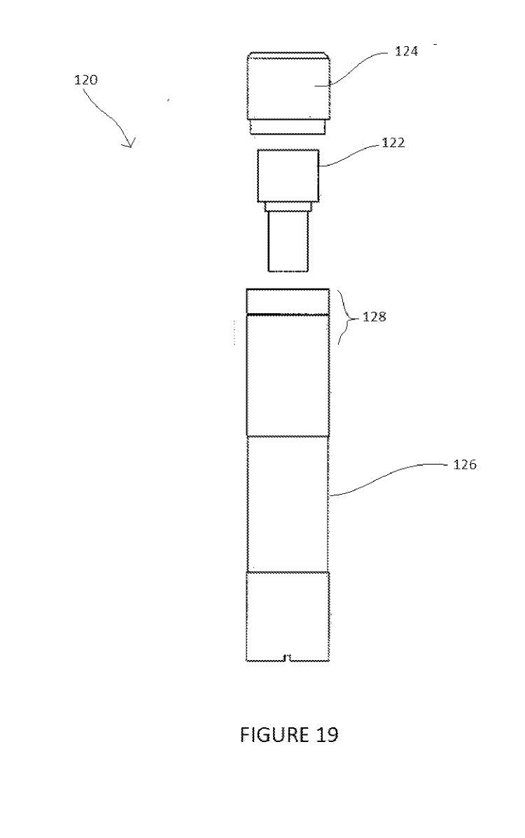

In yet another embodiment of the variable valve/throttled flow aspect of the invention, illustrated in FIG. 19, a complete fluid delivery system 120 is illustrated schematically. Fluid delivery system 120 includes a fluid-permeable cartridge component 122, which may either be a cartridge with an integral valve seat or a cartridge housing with an integral valve seat containing a cartridge therein, as illustrated and described in the foregoing embodiments, or some other equivalent structure, a distal cartridge housing or casing cover section 124, and a breathable fluid delivering base section 126 of the device having a proximal cartridge housing cover section 128 and a valve needle (not shown) included therein. Base section 126 thus combines fluid delivery components such as a propylene glycol or glycerin tank (not shown) and a heating coil or vibrating element (not shown) with variable valve and cartridge retaining components or elements as described above, in a single body. Base section 126 may simply be the product of connecting a coupling adaptor such as coupling adaptor 86, needle valve component/alternative coupling adaptor 86′, or an equivalent component or combination of components into a body including breathable fluid delivery components. Alternatively, base section 126 may include such valve and cartridge retaining components permanently integrated with part or all of the fluid delivery portion of the device.

While the invention has been described with respect to certain embodiments, as will be appreciated by those skilled in the art, it is to be understood that the invention is capable of numerous changes, modifications and rearrangements, and such changes, modifications and rearrangements are intended to be covered by the following claims.

What is claimed is:

1. An apparatus for delivering a functional liquid in a breathable fluid stream for inhalation comprising
a mouthpiece including a distal opening in fluid communication with a proximal opening; and
a cartridge comprising a fluid-permeable packing material, at least a relatively distal portion of the cartridge disposed in fluid communication with the proximal opening of the mouthpiece and at least a relatively proximal portion of the cartridge disposed in fluid communication with a breathable base fluid, the cartridge configured to permit a vacuum applied at the distal mouthpiece opening to draw a fluid stream comprising the breathable base fluid through the packing material, into the mouthpiece through the proximal mouthpiece opening, and out of the mouthpiece through the distal mouthpiece opening; and
the packing material containing a functional liquid disposed to be introduced into the fluid stream as the fluid stream passes through the packing material and to be carried in the fluid stream in a breathable form when the fluid stream passes out of the mouthpiece through the distal opening.

2. The apparatus of claim 1, the cartridge further comprising a casing, at least a portion of the casing surrounding at least a portion of the packing material in radial directions normal to a proximal-to-distal axis.

3. The apparatus of claim 3, the portion of the casing that radially surrounds the portion of the packing material being fluid impermeable.

4. The apparatus of claim 4, the casing further including at least one proximal or distal end, the proximal or distal end being fluid-permeable and retaining the packing material.

5. The apparatus of claim 4, the casing further including a proximal and a distal end, the proximal and distal ends being fluid permeable and sealable by a removably adhered film.

6. The apparatus of claim 1, further comprising a housing containing the cartridge, the housing including a proximal opening in fluid communication with the breathable base fluid and a distal opening in fluid communication with the proximal mouthpiece opening, and the housing including structural features configured to support the cartridge in a position in which a portion of the cartridge is in fluid communication with the proximal housing opening and a portion of the cartridge is in fluid communication with the distal housing opening.

7. The apparatus of claim 7, the mouthpiece being removably connected to the housing adjacent the distal housing opening.

8. The apparatus of claim 7, the housing being removably connected to a fluid delivery device configured to deliver a stream of the breathable base fluid into the proximal housing opening.

9. The apparatus of claim 9, further comprising a coupling adaptor removably connected to the housing adjacent the proximal housing opening and to a connector of the fluid delivery device, from which connector a mouthpiece has been removed.

10. The apparatus of claim 7, the cartridge being manually removably contained in the housing, further comprising a housing cover for releasably locking the cartridge in the housing.

11. The apparatus of claim 11, the housing cover comprising two separate cover sections removably connected together to at least substantially surround the housing but for a proximal housing cover opening in fluid communication with the proximal housing opening and a distal housing cover opening in fluid communication with the distal housing opening.

12. The apparatus of claim 12, the housing cover sections being threaded together.

13. The apparatus of claim 12, the housing cover sections mating together by an insertable portion of one of the sections being slidingly inserted into a receiving portion of the other section.

14. The apparatus of claim 13, further comprising a resilient member disposed on at least one of the insertable portion and the receiving portion to seal the connection between the housing sections and to frictionally retain the housing sections in a mated configuration.

15. The apparatus of claim 7, further comprising
a flow channel in which the packing material is disposed, the flow channel including a wider portion adjacent at least one of the distal and the proximal end of the cartridge, and a narrower portion adjacent the wider portion and extending therefrom in a direction away from the cartridge; and
a spacer abutting the cartridge to maintain an axial distance between the cartridge and the narrower channel portion.

16. A fluid-permeable cartridge for breathable delivery of a functional liquid comprising
a fluid-permeable packing material containing an absorbed or adsorbed quantity of a functional liquid configured to be introduced into a breathable fluid stream flowing through the cartridge.

17. The cartridge of claim 15, further comprising a fluid-impermeable covering disposed over at least a portion of the outer surface area of the packing material.

18. The cartridge of claim 16, the covering comprising a wall structure generally surrounding the packing material.

19. The cartridge of claim 17, the covering further comprising two fluid-permeable ends joined to the wall structure.

20. The cartridge of claim 18, further comprising fluid-impermeable film material removably fixed over the ends of the covering to seal the packing material and functional liquid within the casing for storage and/or transport prior to use of the cartridge.

21. The cartridge of claim 19, the film material being one of metal foil and plastic film and being removably fixed over the ends of the covering by one of adhesive and heat-fused wax.

22. The cartridge of claim 15, the packing material being selected from the group consisting of cotton, foam, fibrous media, stacked thread, stone, and synthetic porous media.

23. A conduit assembly for a breathable fluid delivery device comprising
a fluid-permeable cartridge disposed in a main fluid channel, the cartridge containing a functional liquid disposed to be introduced in a breathable form into a breathable base fluid flowing in the main fluid channel through the cartridge;
a source of the breathable base fluid in fluid communication with an upstream end of the cartridge;
a mouthpiece in fluid communication with a downstream end of the cartridge; and
an adjustable valve system for adjusting the flow impedance of a bypass channel relative to the flow impedance of a portion of the main channel extending through the cartridge, the bypass channel connecting a portion of the main channel upstream of the cartridge to a portion of the main channel downstream of the cartridge.

24. The conduit assembly of claim 22, the adjustable valve system comprising
a tube in fluid communication with the breathable base fluid source; and
a tube in fluid communication with the upstream end of the cartridge;
one of the tubes being an insertable tube comprising a tapered tip narrowing to a smallest exterior cross section at an open end of the tube, at least a portion of the tapered tip being insertable into an open end of the other tube, the other tube being a receiving tube, and
the tubes being relatively movable for insertion and withdrawal of the tapered tip into and out of the receiving tube to vary the cross-sectional area of a clearance between the receiving tube opening and the tapered tip, the bypass channel being in fluid communication with the breathable base fluid source only by a passage extending through a clearance between the receiving tube opening and the tapered tip.

25. The conduit assembly of claim 23, the insertion tube including a structural feature configured to sealingly cover the receiving tube opening when the insertion tube is inserted to a closed valve position, to cut off fluid communication between the upstream portion of the main channel and the downstream portion of the main channel through the bypass channel.
